(19) United States
(12) Patent Application Publication
(10) Pub. No.: US 2015/0148739 A1
(43) Pub. Date: May 28, 2015

(54) Simplified Microplegia Delivery System

(71) Applicant: April Marie Radicella, Atlanta, GA (US)

(72) Inventor: April Marie Radicella, Atlanta, GA (US)

(21) Appl. No.: 14/091,540

(22) Filed: Nov. 27, 2013

(51) Int. Cl. A61M 1/36

(52) U.S. Cl. CPC A61M 1/3664; A61M 1/3666; A61M 2205/3334; A61M 2205/505
USPC 604/65

(57) ABSTRACT

Cardioplegia is a mixture of blood and potassium/crystalloid solution administered to protect the myocardium during cardiopulmonary bypass (CPB) procedures. Microplegia is a termed used to describe cardioplegia that uses minimal (non-traditional) amounts of crystalloid to carry the potassium solution. I have designed a microplegia technique that utilizes a syringe driver but is more user-friendly and cost effective.

### BACKGROUND OF THE INVENTION

You can't breathe. There's tightness in your chest, and the pain is radiating to your arm, your jaw, your back. Get to the hospital, because you are probably having a heart attack. Millions of open heart procedures are conducted every year in the United States alone (U.S. Department of Health and Human Services, n.d.). Perfusionists play a special role in open heart surgery; their goal is to create a bloodless, motionless field for the cardiac surgeon to perform repairs.

### The Bloodless

To achieve a bloodless field, the perfusionist operates a heart/lung machine that diverts the entire blood flow from the patient to a machine that functions as the heart (pump) and lung (oxygenator) back to the patient. Blood is taken from the vena cava and returned to the aorta, completely bypassing the heart and lungs [FIG. 1]. This is termed “cardiopulmonary bypass.”

### The Motionless

During cardiopulmonary bypass, the perfusionist administers cardioplegia to protect and preserve the myocardium. From the Greek, cardioplegia translates to “paralysis of the heart” (MedicineNet, Inc, 2013). To achieve a motionless heart, the perfusionist administers a mix of oxygenated blood and a potassium solution directly to the myocardium. The high potassium dose prevents repolarization causing electrical arrest; electrical arrest leads to mechanical arrest. Achieving arrest is critical not only so that the surgeon can operate on a still target, but primarily to protect the myocardium by decreasing metabolic demand from the heart tissue during cardiopulmonary bypass. Suggested further cardioplegia definition can be found at http://tele.med.ru/book/cardiac_anesthesia/text/gr/gr013.htm#gr013topic021.

Common Cardioplegia Techniques

Cardioplegia solution components are determined by the surgeon. Cardioplegia is delivered at various temperatures from ice cold to normothermic. Cardioplegia is given in various blood-to-crystalloid ratios; the most common is 4:1 (4 parts blood to 1 part crystalloid). The crystalloid solution may have various additives, but the medication critical to achieving arrest is potassium.

### The Induction Dose

The initial dose of cardioplegia is a high concentration of potassium (High K⁺) generally referred to as the induction dose.

### The Maintenance Dose

As the case proceeds, a lower concentration of potassium (Low K⁺) is needed to maintain arrest. The potassium concentration may be titrated down so that the patient achieves a normal serum potassium by the end of the cardiopulmonary bypass run. Typical cardioplegia techniques (4:1, 8:1, etc) limit the perfusionist to using only two solutions: a High K⁺ and a Low K.

Microplegia

Microplegia is a term used to refer to a kind of cardioplegia delivery system that uses a dramatically minimal amount of crystalloid to carry the potassium to the heart. A common goal amongst all hospitals is to reduce the amount of blood transfusions since they come with a steep cost, increased negative side effects, and decreasing availability; one way to decrease the necessity for a blood transfusion is to reduce hemodilutional anemia. As a result, perfusionists are continually developing new ways to cut back on the amount of crystalloid used during cardiopulmonary bypass. To put it in perspective, a normal cardiac procedure, using 4:1 cardioplegia, might deliver anywhere from 200 ml to 1500 ml of crystalloid (contributing to hemodilution). In contrast, a microplegia system might deliver anywhere from 5 ml to 40 ml of crystalloid.

Comparable Item on the Market

There is only one “microplegia” system on the market called the Quest MPS. This system uses a series of piston pumps and pressure gauges to deliver microplegia. There are two complaints/problems with the MPS: 1.) The MPS and its components are very expensive. 2.) The MPS is difficult and time consuming to override in the event of a failure.

### Brief Summary of the Invention

Another option to deliver microplegia is to manipulate a syringe driver. There is no standard syringe driver used for this type of cardioplegia delivery. This technique is effective, but it requires extensive calculations and can be difficult to use during a cardiopulmonary bypass run. I have designed a simplified, cost-effective syringe driver [FIGS. 2 and 3].

### Features

1.) The calculations will already be done for the clinician. The syringe driver will have preset flow-based calculations.

2.) The delivery options are limited for ease of use and mimics terminology that clinicians are accustomed to hearing [FIG. 3].

3.) The driver itself will give the perfusionist the ability to easily toggle between four options.
a. High K⁺=The induction dose. Potassium delivered at a rate of 20 meQ/Liter. [FIG. 3A].
b. Low K⁺=The maintenance dose. Delivered at a rate of 10 meQ/Liter. [FIG. 3B].
c. Burst=A shot of High K⁺ used to quiet electrical activities [FIG. 3C].
d. Blood Only=No potassium is delivered in this mode [FIG. 3D].

4.) This simplified syringe driver is inexpensive and will give perfusionists the ability to deliver microplegia at a reduced cost when compared to the Quest MPS.

5.) The flow semsor [FIG. 2D] will also enable the driver to auto start/stop, eliminating that extra step for the clinician.

6.) This simplified syringe driver system is can be adapted to any current cardioplegia system. This gives the perfusionist the option to use microplegia [FIG. 2B] or 4:1 cardioplegia [FIG. 2A]. This adaptability makes it so the perfusionist can meet surgeon preferences and provides an immediate second option in case of a device failure.

7.) The syringe driver fluid is delivered through a pressure line [FIG. 2F] and cut into the existing cardioplegia line with a 3/16- 3/16 straight connector with a leur lock [FIG. 2E]. This cut-in can be anywhere on the cardioplegia circuit. Preferred location is on the sterile field.

8.) The flow sensor [FIG. 2D] will be placed on the outflow of the cardioplegia heat exchanger [FIG. 2C]. This sensor will monitor the blood flow and communicate to the syringe driver [FIG. 2B].

Considering the rising costs and risks associated with blood transfusions associated with hemodilution during cardioplulmonary bypass, there is an immense need for an inexpensive, user-friendly microplegia system. Perfusionist, surgeons and those that specialize in perfusion will understand the use and adaptability of this system. This syringe driver description is not intended to limit the final product. There may be alterations made to this model.

### BRIEF EXPLANATION OF DRAWINGS AND FIGURES

FIG. 1. This is a diagram of the entire heart lung machine. This shows the flow of blood and several functions. Blood travels from the vena cava, to the blood reservoir, to the pump, to the oxygenator/heat exchanger, then travels to the aorta. A portion from the oxygenated blood travels to the cardioplegia system for myocardial delivery; that is the highlighted area. There can be variations in the design. FIG. 2. This is a drawing that focuses on the cardioplegia circuit. Here we see the oxygenated blood traveling to the cardioplegia roller pump. There are two bags [FIG. 2A] (a High K⁺ and a Low K⁺) of cardioplegia solution that is mixed with the blood (in either a 4:1 or 8:1 ratio). This line can be clamped [FIG. 2G] out so only blood would travel through the system. The blood travels to a heat exchanger [FIG. 2C] and on to the patient. As an alternative to using the bags of cardioplegia [FIG. 2A], this design allows the clinician to connect to the circuit by cutting a 3/16^{th}- 3/16^{th} straight connector with a leur lock [FIG. 2E]. The syringe driver [FIG. 2B] pumps a solution through a pressure line [FIG. 2F] that is connected to that 3/16^{th}- 3/16^{th} connector [FIG. 2E]. Finally, a flow sensor [FIG. 2D] that will communicate with the syringe driver [FIG. 2B] is connected to the outlet of cardioplegia pump.

FIG. 3. This is a close up view how the simplified syringe driver will appear. The top display [FIG. 3F] provides pertinent information such as total volume delivered, current flow rate, etc. There are also 4 options with press buttons beside them [FIGS. 3, A,B,C,D]. The syringe driver is attached to and controls a syringe with a potassium solution [FIG. 3E]. The clinician must indicate which delivery option they want to use. This design could also be a touch screen.

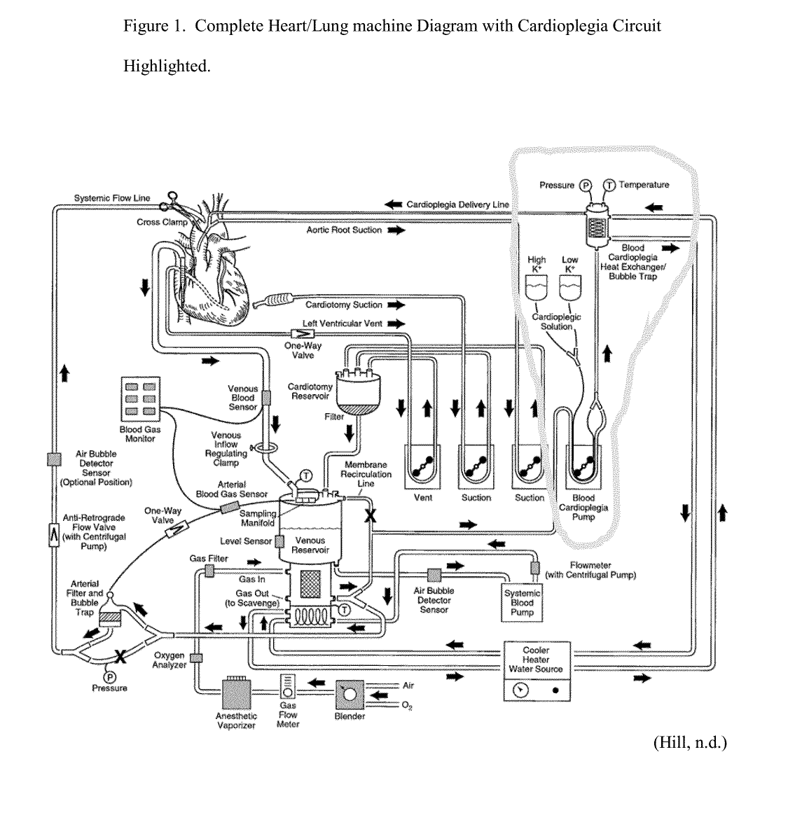

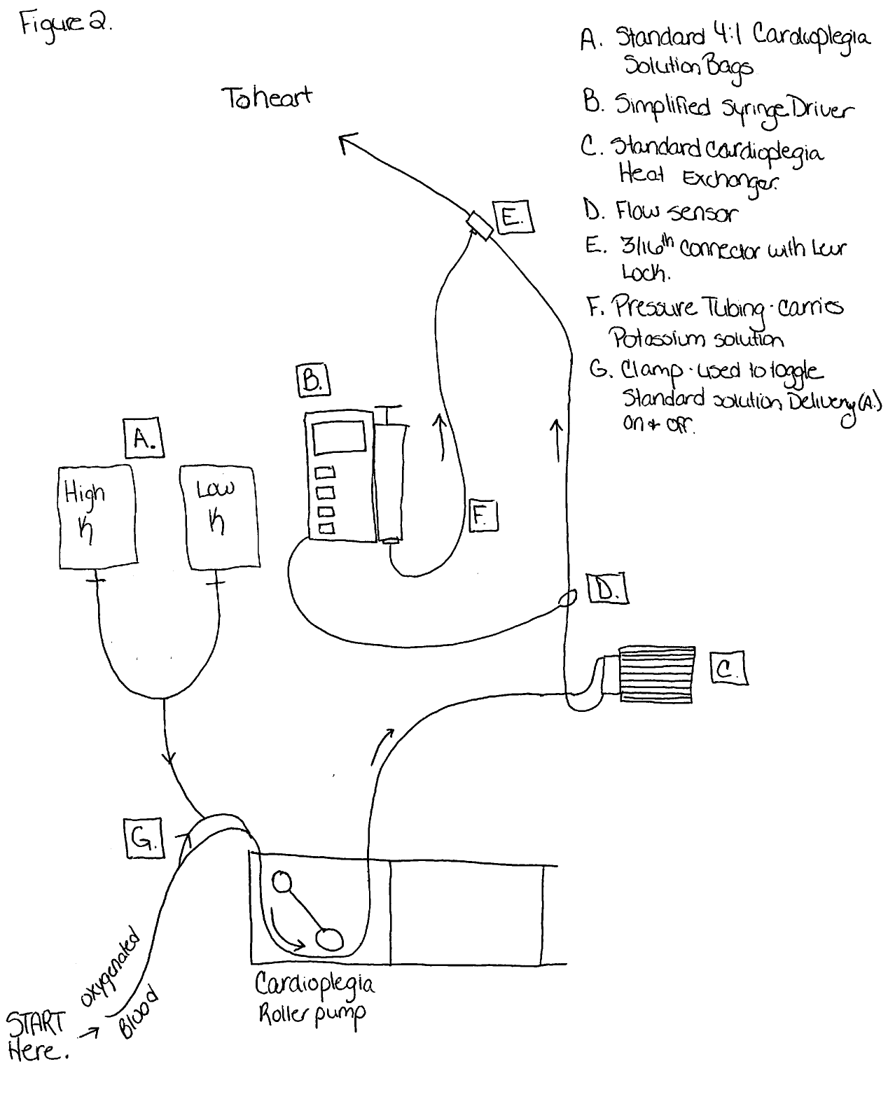

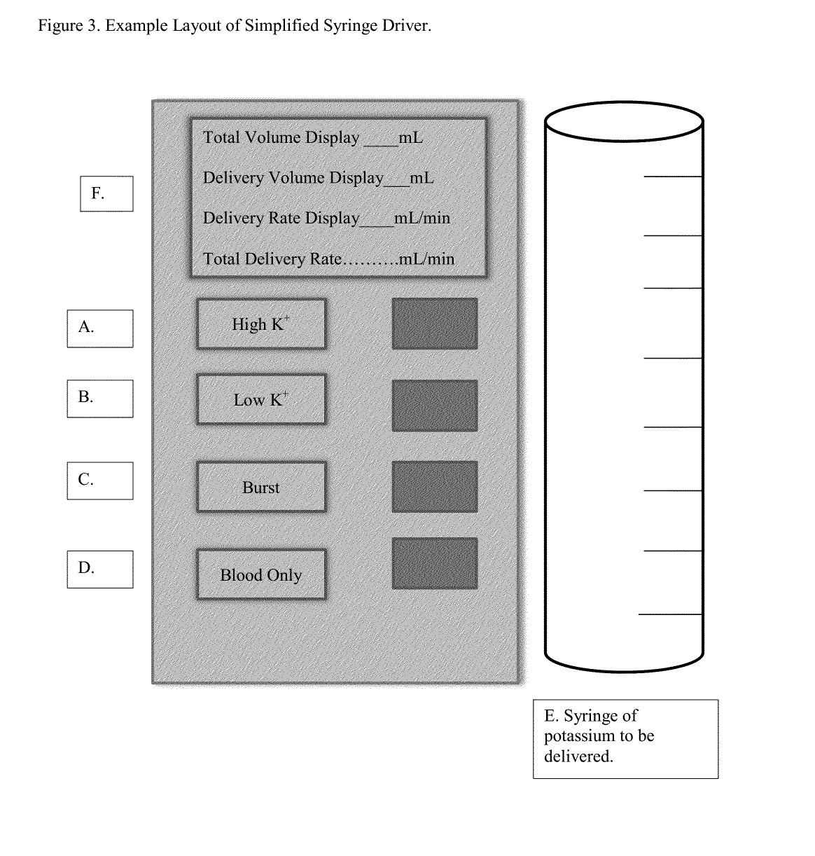

### References

MedicineNet, Inc. (2013, November). Definition of Plegia. Retrieved from http://www.medterms.com/script/main/art.asp?articlekey=26809

U.S. Department of Health and Human Services. (n.d.). Heart Surgery. Retrieved from http://www.nhlbi.nih.gov/health//dci/Diseases/hs/hs_all.html

Hill, Aaron. (n.d.). Retrieved from http://tele.med.ru/book/cardiac_anesthesia/text/gr/gr005.htm

1. The delivery options are limited to 4 options. High K⁺, Low K⁺, Burst, and Blood Only. The labels may have a different name, but the function stays the same. The actual solution may vary by surgeon preference. The delivery rate of 20 meQ/L for induction and 10 meQ/L is a suggested rate but may alter on the final product depending on physician preference.

2. The display on the screen will show pertinent information, and may be customized to each institution.

3. The flow sensor communicates with the syringe driver to auto-start, auto-stop, and determine flow delivery rate based on the actual delivery rate in mL/minute.

4. The syringe driver display may be a touch screen or have buttons to depress.
